(19) United States
(12) Patent Application Publication
(10) Pub. No.: US 2007/0213652 A1
(43) Pub. Date: Sep. 13, 2007

(54) SYSTEM AND METHOD FOR REMOTE BASED CONTROL OF AN IONTOPHORESIS DEVICE

(75) Inventor: Darrick Carter, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: TRANSCUTANEOUS TECHNOLOGIES INC., Tokyo (JP)

(21) Appl. No.: 11/617,609

(22) Filed: Dec. 28, 2006

Related U.S. Application Data
(60) Provisional application No. 60/755,250, filed on Dec. 30, 2005.

(51) Int. Cl. A61N 1/30

(52) U.S. Cl. 604/20

(57) ABSTRACT

The present invention comprises an active agent reservoir that stores a quantity of an active agent, an active electrode element operable to apply an electrical potential of a first polarity to deliver at least a portion of the active agent to a biological interface from the iontophoresis device, and a communications port providing a communications path between the iontophoresis device and a remote device. A method of the invention comprises operating an interface system to remotely interact with iontophoresis devices useful in delivery of active agents to biological interfaces including providing a user interface at a location remote from at least some of the iontophoresis devices, communicating with at least two of the iontophoresis devices, and providing information about the iontophoresis devices via the user interface.

### CROSS-REFERENCE TO RELATED APPLICATION

This application claims the benefit under 35 U.S.C. § 119(e) of U.S. Provisional Patent Application No. 60/755,250 filed Dec. 30, 2005, which provisional application is incorporated herein by reference in its entirety.

### BACKGROUND OF THE INVENTION

1. Field of the Invention

This disclosure generally relates to the field of iontophoresis, and more particularly to the remote based controlling and/or monitoring of the delivery of an active agent, such as therapeutic agents or drugs, to a biological interface under the influence of electromotive force.

2. Description of the Related Art

Iontophoresis employs an electro-motive force to transfer an active agent such as an ionic drug or other therapeutic agent to a biological interface such as skin or mucus membrane.

Iontophoresis devices typically include an active electrode assembly and a counter electrode assembly, each coupled to opposite poles or terminals of a power source, such as a chemical battery. Each electrode assembly typically includes a respective electrode element to apply an electromotive force. Such electrode elements often comprise a sacrificial element or compound, for example silver or silver chloride.

The active agent may be either cation or anion, and the power source can be configured to apply the appropriate voltage polarity based on the polarity of the active agent. Iontophoresis may be advantageously used to enhance the delivery rate of the active agent. The active agent may be stored in a reservoir such as a cavity, or stored in a porous structure or as a gel. As discussed in U.S. Pat. No. 5,395,310, an ion exchange membrane may be positioned to serve as a polarity selective barrier between the active agent reservoir and the biological interface.

It is time consuming and costly to have medical personnel and practitioners physically available to clock, maintain charts, monitor and update the operation of each iontophoresis device. Further, it has been recognized that allowing patients some control over certain medication such as, for example pain relief medication, provides psychological benefits by empowering the patient, as well as reducing the required staff to patient ratio.

It is often time consuming and costly for the patient to remain within a vicinity of a medical practitioner so that the practitioner may, for example, control the amount of active agent to be delivered and monitor the delivery of the active agent. It may results in lengthy hospital stays costing thousands of dollars a day. Additionally, due to a limitation in patient capacity users of the iontophoresis device may be taking the place of others who require the presence of medical practitioners for the treatment of certain ailments.

Therefore, it may be desirable to have an iontophoresis system that addresses one or more of these problems.

### BRIEF SUMMARY OF THE INVENTION

In one embodiment, an iontophoresis device comprises an active agent reservoir that stores a quantity of an active agent, an active electrode element operable to apply an electrical potential of a first polarity to deliver at least a portion of the active agent to a biological interface from the iontophoresis device, and a communications port providing a communications path between the iontophoresis device and a remote device.

In another embodiment, an iontophoresis interface system comprises a user interface operable by a user to produce user generated requests and to provide information to the user, a communications port providing a communications path between the iontophoresis interface system and at least one iontophoresis device remotely located with respect to the iontophoresis interface system, and a processor coupled to control communications with the at least one iontophoresis device via the communications port based at least in part on the user generated requests.

In yet another embodiment, an iontophoresis system comprises at least a first iontophoresis device operable to deliver an active agent to a biological interface, the first iontophoresis device including an active agent reservoir that stores a quantity of the active agent and an active electrode element operable to apply an electrical potential of a first polarity to deliver at least a portion of the active agent to the biological interface from the iontophoresis device, and an iontophoresis interface system remotely located with respect to the first iontophoresis device, the iontophoresis interface system comprising a user interface operable by a user to produce user generated requests and to provide information to the user, a communications port providing a communications path between the iontophoresis interface system and the at least one iontophoresis device, and a processor coupled to control communications with the at least one iontophoresis device via the communications port based at least in part on the user generated requests.

In a further embodiment, a method of operating an iontophoresis device comprises storing a quantity of an active agent in an active agent reservoir, delivering at least a portion of the active agent to a biological interface from the iontophoresis device by applying an electrical potential of a first polarity to an active electrode element, and communicating between the iontophoresis device and a remote device via a communications port.

In another further embodiment, a method of operating an interface system to remotely interact with iontophoresis devices useful in delivery of active agents to biological interfaces including providing a user interface at a location remote from at least some of the iontophoresis devices, comprises communicating with at least two of the iontophoresis devices, and providing information about the iontophoresis devices via the user interface.

In yet a further embodiment, a method of operating an iontophoresis system comprises providing communications between a plurality of iontophoresis devices and at least one iontophoresis interface system remotely located from the iontophoresis devices, operating the iontophoresis devices at least in part on commands received from the iontophoresis interface system, and at least temporarily storing operational condition information at the iontophoresis interface system, the operational condition information indicative of at least one operating parameter for a number of the iontophoresis devices.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic diagram of an iontophoresis system including an iontophoresis interface system and a number of iontophoresis devices, where the iontophoresis interface system communicates with the iontophoresis devices via a communications network, according to one illustrated embodiment.

FIG. 2 is a functional block diagram of a computing system suitable for embodying at least one embodiment of the iontophoresis interface system, according to one illustrated embodiment.

FIGS. 3-6 are sequential schematic representations of a user interface display produced by the iontophoresis interface system to facilitate a practitioner or health care professional in controlling and/or monitoring at least one of the iontophoresis devices, according to one illustrated embodiment.

FIG. 7 is a block diagram of the iontophoresis device comprising active and counter electrode assemblies, a controller, radio and antenna, regulator and power source, according to one illustrated embodiment.

FIG. 8 is a high level flow diagram showing a method of operating the iontophoresis device to monitor and report operational parameters and/or performance information, according to one illustrated embodiment.

FIG. 9 is a low level flow diagram showing the method of monitoring operational parameter information in further detail, according to one illustrated embodiment.

FIG. 10 is a high level flow diagram showing a method of operating the iontophoresis device to receive requests for adjusting at least one operational parameter and modifying the active agent delivery in response thereto, according to one illustrated embodiment.

FIG. 11 is a high level flow diagram showing a method of operating the iontophoresis device to provide authorizing requests to the iontophoresis interface system and/or remote device based on user generated operational requests, and responding to commands provided from the iontophoresis interface system and/or remote device in response to the authorizing requests, according to one illustrated embodiment.

FIG. 12 is a low level flow diagram showing a method of adjusting the active agent delivery regime based on the at least one operational parameter information in further detail, according to one illustrated embodiment.

FIG. 13 is a high level flow diagram showing a method of operating the iontophoresis interface system to communicate commands for monitoring operational parameter information to the respective iontophoresis devices, according to one illustrated embodiment.

FIG. 14 is a high level flow diagram showing a method of operating the iontophoresis interface system to communicate commands for modifying operational parameter information to the respective iontophoresis devices, according to one illustrated embodiment.

FIG. 15 is a high level flow diagram showing a method of operating the iontophoresis interface system to communicate commands for modifying operational parameter information in response to authorization requests received from respective iontophoresis devices, according to one illustrated embodiment.

FIG. 16 is a high level flow diagram showing a method of operating the iontophoresis system to at least partially control the active agent delivery of the iontophoresis device, according to one illustrated embodiment.

FIG. 17 is a high level flow diagram showing a method of operating the iontophoresis system to monitor the active agent delivery of the iontophoresis device, according to one illustrated embodiment.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, radios, networks or embedded controllers, for example oscillators, accumulators, counters, and comparators have not been described in detail so as not to obscure the embodiments of the present invention.

Unless the context requires otherwise, throughout the specification and claims which-follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used herein and in the claims, the term “membrane” means a layer, barrier or material, which may, or may not be permeable. Unless specified otherwise, membranes may take the form of a solid, liquid or gel, and may or may not have a distinct lattice or cross-linked structure.

As used herein and in the claims, the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein and in the claims, the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. Examples of commercially available cation exchange membranes include those available under the designators NEOSEPTA, CM-1, CM-2, CMX, CMS, and CMB from Tokuyama Co., Ltd. Examples of commercially available anion exchange membranes include those available under the designators NEOSEPTA, AM-1, AM-3, AMX, AHA, ACH and ACS also from Tokuyama Co., Ltd.

As used herein and in the claims, the term bipolar membrane means a membrane that is selective to two different charges or polarities. Unless specified otherwise, a bipolar membrane may take the form of a unitary membrane structure or multiple membrane structure. The unitary membrane structure may have a first portion including cation ion exchange material or groups and a second portion opposed to the first portion, including anion ion exchange material or groups. The multiple membrane structure (e.g., two film) may be formed by a cation exchange membrane attached or coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that substantially selective based on a size or molecular weight of the ion. Thus, a semi-permeable membrane substantially passes ions of a first molecular weight or size, while substantially blocking passage of ions of a second molecular weight or size, greater than the first molecular weight or size.

As used herein and in the claims, the term “porous membrane” means a membrane that is substantially selective with respect to ions at issue. For example, a porous membrane is one that is not substantially selective based on polarity, and not substantially selective based on the molecular weight or size of a subject element or compound.

A used herein and in the claims, the term “reservoir” means any form of mechanism to retain an element or compound in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound.

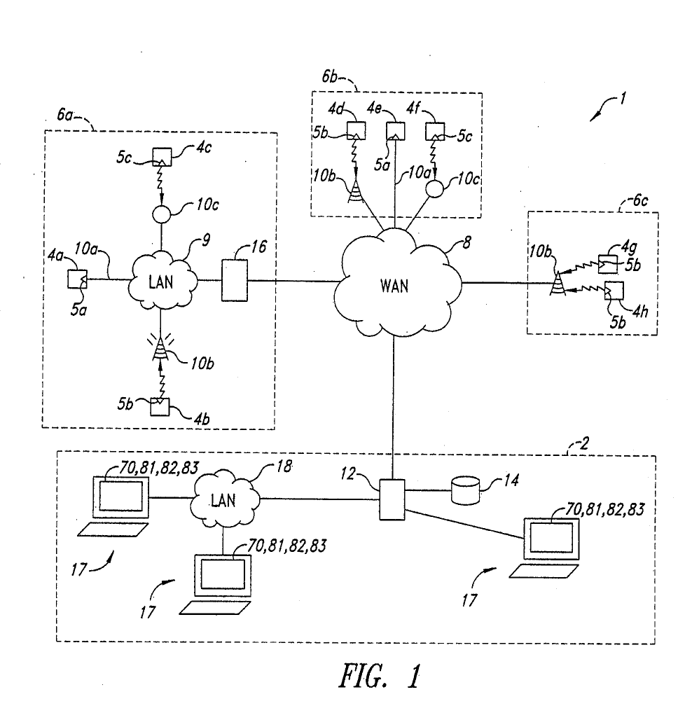

The following discussion and FIG. 1 provide a brief, general description of a suitable computing environment in which some illustrated embodiments may be implemented. Although not required, embodiments will be described in the general context of computer-executable instructions, such as program application modules, objects, or macros being executed by a personal computer. Those skilled in the relevant art will appreciate that some embodiments can be practiced with other computing system configurations, including handheld devices, multiprocessor systems, microprocessor-based or programmable consumer electronics, network PCs, minicomputers, mainframe computers, and the like. Some embodiments can be practiced in distributed computing environments where tasks or modules are performed by remote processing devices, which are linked through a communications network. In a distributed computing environment, program modules may be located in both local and remote memory storage devices.

As an overview, an embodiment provides communication between a plurality of iontophoresis devices and an iontophoresis interface system, located at a remote location relative the iontophoresis devices wherein the iontophoresis interface system has the ability to control and/or monitor active agent delivery from the iontophoresis device to a biological interface. For example, a user of the iontophoresis device may be a patient receiving controlled doses of an active agent via iontophoresis, to alleviate pain, cure a disease and/or diagnose a condition. In the event that the patient has received all the needed medical procedures and is in the recovery stage, the patient may be kept under observation in a medical facility for the sole purpose of monitoring and/or controlling the drug delivery, thereby prolonging the patient's stay in the medical facility. In such circumstances costly medical resources are being used for merely controlling and/or monitoring active agent delivery.

The iontophoresis system allows medical practitioners to monitor and/or control the active agent delivery for several iontophoresis devices through an interface that may be located at a location remote from the iontophoresis devices. The interface may receive requests for increased or decreased dosage from each one of the users of the iontophoresis devices but may require the authorization of the medical practitioner or health care professional to adjust the dosage. The interface may further comprise a server that is programmed to automatically allow a predetermined change in dosage for each one of the users, upon receiving the request from the user. The iontophoresis system thereby eliminates the need for the user to be checked into a costly medical facility and allows the medical practitioner to allocate more time for patients undergoing medical procedures that require the presence and/or participation of the practitioner or health care professional.

FIG. 1 shows an iontophoresis system 1 comprising at least one iontophoresis interface system 2 and a number of iontophoresis devices 4a-4h (collectively 4), remotely located with respect to the iontophoresis interface system 2. Each of the plurality of iontophoresis devices 4 is operable to deliver an active agent to a biological interface. Each of the iontophoresis devices 4 has a respective communications port, of which three types are illustrated. A first type of communications port is a connector 5a that allows a wired communications connection to be made to the respective iontophoresis device 4a, 4e. A second type of communications port is an antenna 5b that allows an RF wireless communications connection to be made to the respective iontophoresis device 4b, 4d, 4g, 4h. As used herein and in the claims the term RF or radio includes electromagnetic energy with wavelengths longer than that of visible light or infrared (e.g., radio, microwave, etc.). A third type of communications port is an optical transceiver 5c to allow an optical wireless communications connection to be made with the iontophoresis device 4c, 4f. As used herein and in the claims the term optical includes electromagnetic energy with wavelengths in the range of light, including visible light, ultraviolet and/or infrared. The communications ports are collectively referenced herein as 5.

The iontophoresis devices 4 may be grouped in one or more iontophoresis device environments 6a-6c (collectively 6), or may be located individually at widely dispersed locations.

Transmission connectors 10a-10c (collectively 10) may couple transmission signals between the iontophoresis devices 4 and the iontophoresis interface system 2 via one or more networks, for example one or more wide area networks (WAN) 8, Wired-Wide Area Network (WWAN), and/or one or more local area networks (LAN) 9. The transmission connectors 10 may comprise a hard wired connection 10a, an antenna connection 10b, an optical transmission connection 10c, or any suitable connection hardware or medium (for sake of clarity, all suitable connection hardware or mediums are referred to by reference number 10) that provides a communications path of at least one of a wired network connection or wireless network connection to and from the iontophoresis device 4a-4c via the associated communications port 5a-5c. The wired connection 10a may be a coaxial cable, fiber optic cable, copper wire or similar wired connection while the antenna connection 10b is appropriate for radio frequency (RF) transmission.

One embodiment of the iontophoresis device environment 6a may comprise the plurality of iontophoresis devices 4a-4c communicatively coupled through their respective communications ports 5a-5c to transmit signals to their respective transmission connectors 10a-10c. The transmission connectors 10 may further transmit the received signals to the iontophoresis device environment network 9 such as for example LAN 9. The LAN 9 may be of Ethernet© specification that handles simultaneous communication commands. The network 9 may further be communicatively coupled to an iontophoresis device environment server 16, the iontophoresis device environment server 16 being operable to receive operational requests from a user of the iontophoresis device 4a-4c and/or information pertaining to the operational conditions of the active agent delivery for each of the iontophoresis devices 4a-4c. The iontophoresis device environment server 16 may be programmed to authorize operational requests based on a predetermined algorithm and may serve as a database 14 for storing information pertaining to the operational conditions of each iontophoresis device 4a-4c. The stored operational conditions of each iontophoresis device 4a-4c may be further communicated to the iontophoresis interface system 2 via the network 8 for monitoring the active agent delivery.

The iontophoresis device environment server 16 may further provide operational commands and/or monitoring commands to the iontophoresis devices 4a-4c for adjusting and/or monitoring at least one operational condition of the device 4a-4c. The operational and/or monitoring commands may be initiated by the iontophoresis interface system 2 through the network 8. The server 16 may transmit the appropriate signals through the LAN 9 and transmit via one or more of the transmission connectors 10a-10c to the desired iontophoresis device 4a-4c.

In another embodiment, the iontophoresis device environment 6b comprises one or more iontophoresis devices 4d-4f communicatively coupled through communications ports 5a-5c to transmit signals to the respective transmission connectors 10a-10c. The transmission connectors 10 further transmit the signals received from the iontophoresis device 4d-4f to the iontophoresis interface system 2 via the iontophoresis system network 8 (e.g., WAN or WWAN). The iontophoresis devices 4d-4f may also receive communication requests and/or commands from the iontophoresis interface system 2 via the network 8 and through the appropriate transmission connector 10a-10c and associated communications port 5a-5c.

In yet another embodiment, the iontophoresis device environment 6c comprises a plurality of iontophoresis devices 4g, 4h communicatively coupled through communications ports 5b to transmit signals to a common transmission connector 10b. The transmission connector 10b further transmits the received signals to the iontophoresis interface system 2 via the network 8. The iontophoresis devices 4g, 4h may also receive communication requests and/or commands from the iontophoresis interface system 2 via the network 8 and through the common transmission connector 10b and associated communications port 5b.

The iontophoresis interface system 2 remotely located with respect to the iontophoresis device environments 6 comprises a plurality of computing systems 17 communicatively coupled to a server 12. The server 12 is communicatively coupled to the network 8 to receive the requests and/or operational conditions from the iontophoresis device 4, and may also include a database 14 to store the requests and/or operational conditions. The database 14 may be accessed through the server 12.

The computing systems 17 may communicate through a network 18 such as, for example, a LAN 18 and retrieve stored information from the database 14. The server 12 allows for computing systems 17 in the network 18 to have a shared resource. The iontophoresis interface system 2 may communicate commands to the iontophoresis devices 4 via the WAN 8 or any other network that communicatively connects the iontophoresis devices 4 to the iontophoresis interface system 2. The commands may comprise adjusting the active agent delivery and/or requesting the monitoring of operational conditions. As described above, the database 14 may store operational conditions pertaining to each iontophoresis device 4a-4h and authorization requests for adjusting active agent delivery dosage. The medical practitioner may provide the authorization for adjusting at least one operational aspect of the active agent delivery via one of the computing systems 17 and/or may program the iontophoresis device environment server 16 to authorize specific user requests. The operational conditions stored in the database 14 may be retrieved for monitoring via one of the computer systems 17, through the server 12. Each of the computer systems 17 comprises a user interface 46, 50 configured to allow for the medical practitioner and/or health care professional to communicate with the desired iontophoresis device 4 and/or retrieve information via the server 12, as described above.

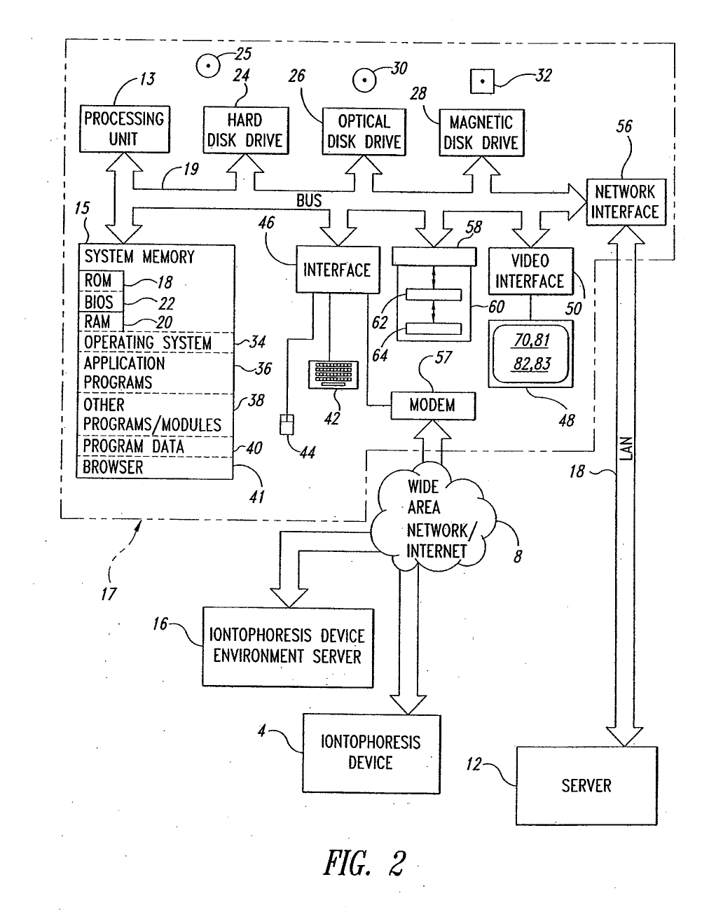

FIG. 2 shows a functional block diagram of the computing system 17 suitable for embodying at least one embodiment of the iontophoresis system 1.

The computing system 17 includes a processor unit 13, a system memory 15 and a system bus 19 that couples various system components including the system memory 15 to the processing unit 13. The processing unit 13 may be any logical processing unit, such as one or more central processing units (CPUs), digital signal processors (DSPs), application-specific integrated circuits (ASIC), etc. Unless described otherwise, the construction and operation of the various blocks shown in FIG. 2 are of conventional design. As a result, such blocks need not be described in further detail herein, as they will be understood by those skilled in the relevant art.

The system bus 19 can employ any known bus structures or architectures, including a memory bus with memory controller, a peripheral bus, and/or a local bus. The system memory 15 includes read-only memory (“ROM”) 18 and random access memory (“RAM”) 20. A basic input/output system (“BIOS”) 22, which can form part of the ROM 18, contains basic routines that help transfer information between elements within the computing system 17, such as during startup.

The computing system 17 also includes one or more spinning media memories such as a hard disk drive 24 for reading from and writing to a hard disk 25, and an optical disk drive 26 and a magnetic disk drive 28 for reading from and writing to removable optical disks 30 and magnetic disks 32, respectively. The optical disk 30 can be a CD-ROM, while the magnetic disk 32 can be a magnetic floppy disk or diskette. The hard disk drive 24, optical disk drive 26 and magnetic disk drive 28 communicate with the processing unit 13 via the bus 16. The hard disk drive 24, optical disk drive 26 and magnetic disk drive 28 may include interfaces or controllers coupled between such drives and the bus 16, as is known by those skilled in the relevant art, for example via an IDE (i.e., Integrated Drive Electronics) interface. The drives 24, 26 and 28, and their associated computer-readable media, provide nonvolatile storage of computer-readable instructions, data structures, program modules and other data for the computing system 17. Although the depicted computing system 17 employs hard disk 25, optical disk 30 and magnetic disk 32, those skilled in the relevant art will appreciate that other types of spinning media memory computer-readable media may be employed, such as, digital video disks (“DVD”), Bernoulli cartridges, etc. Those skilled in the relevant art will also appreciate that other types of computer-readable media that can store data accessible by a computer may be employed, for example, non-spinning media memories such as magnetic cassettes, flash memory cards, RAMs, ROMs, smart cards, etc.

Program modules can be stored in the system memory 15, such as an operating system 34, one or more application programs 36, other programs or modules 38, and program data 40. The system memory 15 also includes a browser 41 for permitting the computing system 17 to access and exchange data with sources such as websites of the Internet, corporate intranets, or other networks, as well as other server applications on server computers. The browser 41 is markup language based, such as hypertext markup language (“HTML”), and operate with markup languages that use syntactically delimited characters added to the data of a document to represent the structure of the document.

While shown in FIG. 2 as being stored in the system memory, the operating system 34, application programs 36, other program modules 38, program data 40 and browser 41 can be stored on the hard disk 25 of the hard disk drive 24, the optical disk 30 and the optical disk drive 26 and/or the magnetic disk 32 of the magnetic disk drive 28. A user such as, for example, the health care professional can enter commands and information to the computing system 17 through input devices such as a keyboard 42 and a pointing device such as a mouse 44. Other input devices can include a microphone, joystick, game pad, scanner, etc. These and other input devices are connected to the processing unit 13 through an interface 46 such as a serial port interface that couples to the bus 16, although other interfaces such as a parallel port, a game port or a universal serial bus (“USB”) can be used. A monitor 48 or other display devices may be coupled to the bus 16 via video interface 50, such as a video adapter. The computing system 17 can include other output devices such as speakers, printers, etc.

The computing system 17 can operate in a networked environment using logical connections to one or more remote computers or the server 12. The computing system 17 may employ any known means of communications, such as through a local area network (“LAN”) or a wide area network (“WAN”) or the Internet. Such networking environments are well known in enterprise-wide computer networks, intranets, and the Internet.

When used in a LAN networking environment, the computing system 17 is connected to the LAN 18 through an adapter or network interface 56 (communicatively linked to the bus 19). When used in a WAN networking environment, the computing system 17 often includes a modem 57 or other device for establishing communications over the WAN/Internet 8. The modem 57 is shown in FIG. 2 as communicatively linked between the interface 46 and the WAN/Internet 8. In a networked environment, program modules, application programs, or data, or portions thereof, can be stored in a server computer (not shown). Those skilled in the relevant art will readily recognize that the network connections shown in FIG. 2 are only some examples of establishing communication links between computers, servers 12, and/or iontophoresis devices 4 and other links may be used, including wireless links.

The computing system 17 may include one or more interfaces such as slot 58 to allow the addition of devices either internally or externally to the computing system 17. For example, suitable interfaces may include ISA (i.e., Industry Standard Architecture), IDE, PCI (i.e., Personal Computer Interface) and/or AGP (i.e., Advance Graphics Processor) slot connectors for option cards, serial and/or parallel ports, USB ports (i.e., Universal Serial Bus), audio input/output (i.e., I/O) and MIDI/joystick connectors, and/or slots for memory.

The term “computer-readable medium” as used herein refers to any medium that participates in providing instructions to processor unit 13 for execution. Such a medium may take many forms, including but not limited to, non-volatile media, volatile media, and transmission media. Non-volatile media includes, for example, hard, optical or magnetic disks 25, 30, 32, respectively. Volatile media includes dynamic memory, such as system memory 15. Transmission media includes coaxial cables, copper wire and fiber optics, including the wires that comprise system bus 19. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor unit 13 for execution. For example, the instructions may initially be carried on a magnetic disk of a remote computer. The remote computer can load the instructions into its dynamic memory and send the instructions over a telephone line using a modem. A modem 57 local to computer system 11 can receive the data on the telephone line and use an infrared transmitter to convert the data to an infrared signal. An infrared detector coupled to the system bus 19 can receive the data carried in the infrared signal and place the data on system bus 19. The system bus 19 carries the data to system memory 15, from which processor unit 13 retrieves and executes the instructions. The instructions received by system memory 15 may optionally be stored on storage device either before or after execution by processor unit 13.

FIGS. 3-6 show a sequence of exemplary display screens 70, 81, 82, 83 comprising a portion of a user interface 50 presented by the iontophoresis interface system 2 to facilitate the medical practitioner or health care professional in controlling and/or monitoring at least one of the iontophoresis devices 4, according to one illustrative embodiment.

In particular, FIG. 3 shows an initial display screen 70 presented by the iontophoresis interface system 2. The initial display screen 70 includes a dialog box 59 with multiple input fields comprising a username field 65 and a password field 66. The input fields identify the user who is gaining access to the interface system 4. In another embodiment, the user may alternatively or additionally be prompted to provide one or more of a password or personnel identification number (PIN) and/or biometric identification features such as, for example, a fingerprint scan, voice, retinal or iris, facial or hand geometry before access to the iontophoresis interface system 2 is granted.

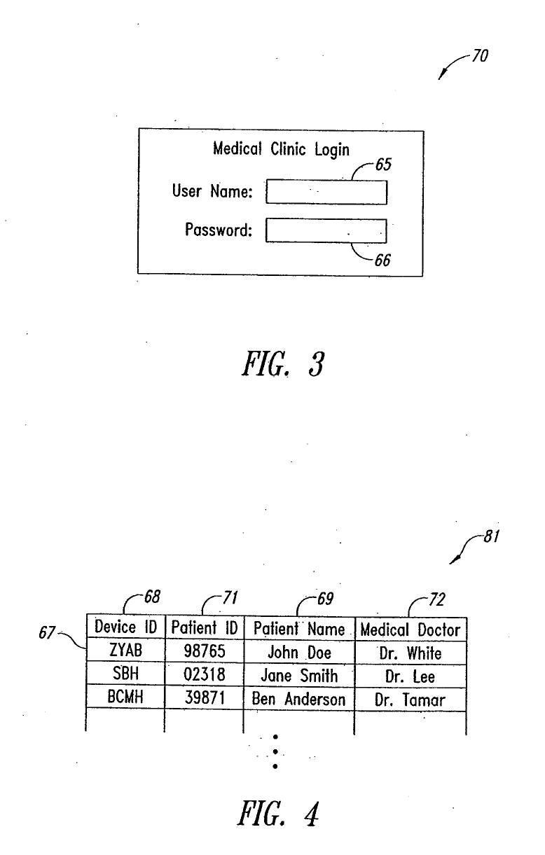

FIG. 4 shows a display screen 81 presented by the iontophoresis interface system 2 that allows the health care professional to identify and access the plurality of iontophoresis devices 4 that are active or that may be activated. The display screen 70 includes a device table 67 comprising a list of the active or activatable iontophoresis devices 4 represented by one or more fields of relevant information associated with each active or activatable device 4. For example, an active or activatable iontophoresis device 4 may be represented by a device identification number 68, patient name 69, patient ID number 71, supervising medical doctor 72, and/or location of the iontophoresis device 4, to name a few representations. The health care professional may identify a desired iontophoresis device 4 for adjusting and/or monitoring at least one operational condition of the iontophoresis device 4 by selecting the corresponding device identifier from the device table 67.

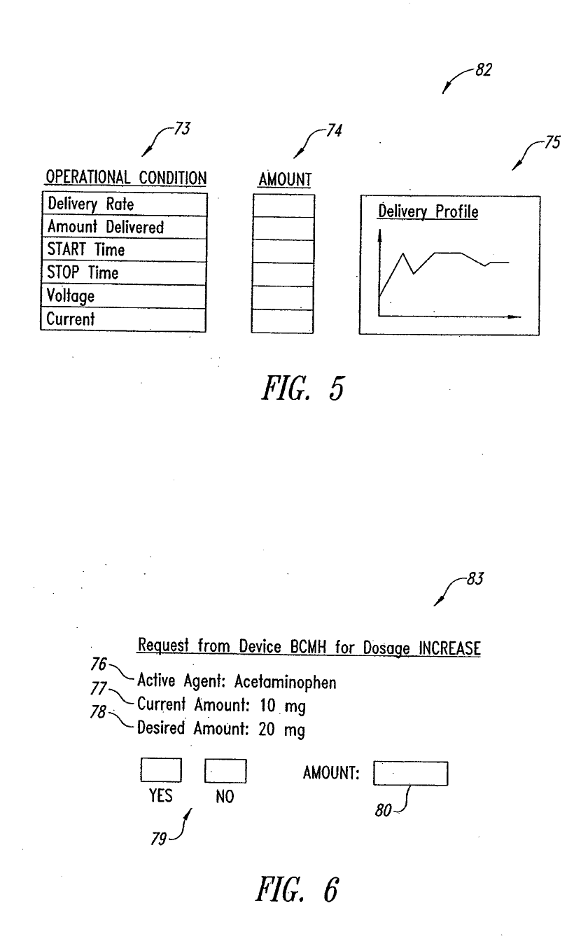

FIG. 5 shows a display screen 82 presented by the iontophoresis interface system 2, that facilitates the adjusting and/or monitoring of at least one operational condition of the iontophoresis device 4. The display screen 82 includes a list of the operational conditions 73 associated with the selected iontophoresis device 4, an adjustment field area 74 for controlling at least one of the selected operational conditions, and a graphics area 75 for depicting a graphical representation of at least one selected operational condition such as, for example, the delivery profile of the active agent. The user or health care professional may select the at least one operational condition for adjustment and/or monitoring from the list of operational conditions 73 and indicate the amount of adjustment to the selected operational condition in the amount field 74.

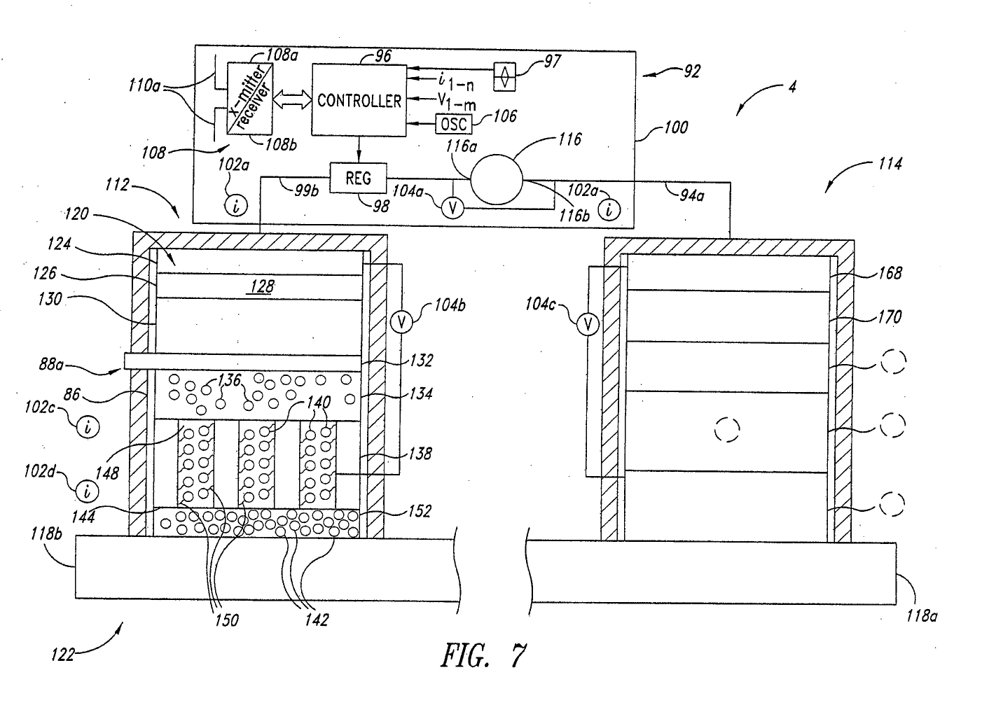

FIG. 6 shows a display screen 83 presented by the iontophoresis interface system 2 in response to a request from at least one iontophoresis device 4. The request may, for example, be a request to adjust a dosage such as, for example, an increase or decrease in active agent dosage. The display screen 83 includes an identification of the iontophoresis device 5 issuing the request, the type of active agent being delivered 76, the current dosage 77, and the desired dosage 78. Additionally, the display screen includes YES and NO icons 79 with an AMOUNT field 80 for specifying the new amount of active agent to be delivered in the event that the user authorizes such a request. The display screen 83 of FIG. 6 may appear as a pop-up window on any of the display screens described above, so as to allow for an immediate response by the health care professional to any such requests.

One skilled in the art will recognize that the display screens 70, 81, 82, 83 generated by the iontophoresis interface system 2 and shown in FIGS. 3-6 may be modified to include additional use interface components and/or information, and may be rearranged into other data and input arrangements.

FIG. 7 shows a block diagram of the iontophoresis device 4 according to one illustrated embodiment. The iontophoresis device comprises active and counter electrode assemblies 112, 114, a controller 96, radio 108 and antenna 108b, regulator 98 and power source 116.

The active electrode assembly 112 may be positioned on or proximate a first portion 118b of a biological interface 118, and the counter assembly 114 may be positioned proximate a second portion 118a of the biological interface 118. Each electrode assembly 112, 114 is electrically coupled to a power source 116 and operable to supply at least one active agent to at least the first portion 118b of the biological interface 118 via iontophoresis, according to one illustrated embodiment. The biological interface 118 may take a variety of forms, for example, a portion of skin, mucous membrane, gum, tooth or other bodily tissue.

In the illustrated embodiment, the active electrode assembly 112 comprises, from an interior 120 to an exterior 122 of the active electrode assembly 112, an active electrode element 124, an electrolyte reservoir 126 storing an electrolyte 128, an inner ion selective membrane 130, an optional inner sealing liner 132, an inner active agent reservoir 134 storing active agent 136, an outermost ion selective membrane 138 that caches additional active agent 140, and further active agent 142 carried by an outer surface 144 of the outermost ion selective membrane 138. The active electrode assembly may include additional components and/or may omit some components. Selected ones of the above elements or structures will be discussed in detail below. More detailed description of these elements, as well as descriptions of the other elements may be found in U.S. patent application Ser. No. 10/488,970, filed Mar. 9, 2004 and Japanese patent application 2004/317317, filed Oct. 29, 2004.

The active electrode element 124 is coupled to a first pole 116a of the power source 116 and positioned in the active electrode assembly 112 to apply an electromotive force or current to transport active agent 136, 140, 142 via various other components of the active electrode assembly 112. The active electrode element 124 may take a variety of forms, for example a sacrificial element or a carbon based electrode element. The inner active agent reservoir 134 may take a variety of forms including any structure capable of temporarily retaining active agent 136, and in some embodiments may even be the active agent 136 itself, for example, where the active agent 136 is in a gel, semi-solid or solid form. For example, the inner active agent reservoir 134 may take the form of a pouch or other receptacle, a membrane with pores, cavities or interstices, particularly where the active agent 136 is a liquid. The inner active agent reservoir 134 may advantageously allow larger doses of the active agent 136 to be loaded in the active electrode assembly 112.

The outermost ion selective membrane 138 may advantageously cache active agent 140. In particular, the ion exchange groups or material 150 temporarily retains ions of the same polarity as the polarity of the active agent in the absence of electromotive force or current and substantially releases those ions when replaced with substitutive ions of like polarity or charge under the influence of an electromotive force or current. Alternatively, the outermost ion selective membrane 138 may take the form of semi-permeable or microporous membrane which is selective by size. In some embodiments, such a semi-permeable membrane may advantageously cache active agent 140, for example by employing a removably releasable outer release liner (not shown) to retain the active agent 140 until the outer release liner is removed prior to use.

The active agent 142 that fails to bond to the ion exchange groups of material 150 may adhere to the outer surface 144 of the outermost ion selective membrane 138 as the further active agent 142. Alternatively, or additionally, the further active agent 142 may be positively deposited on and/or adhered to at least a portion of the outer surface 144 of the outermost ion selective membrane 138, for example, by spraying, flooding, coating, electrostatically, vapor deposition, and/or otherwise. In some embodiments, the active agent 136, additional active agent 140, and/or further active agent 142 may be identical or similar compositions or elements. In other embodiments, the active agent 136, additional active agent 140, and/or further active agent 142 may be different compositions or elements from one another. Other combinations are possible.

The power source 116 may take the form of one or more chemical battery cells, super- or ultra-capacitors, or fuel cells. The power source 116 may, for example, provide a voltage of 12.8V DC, with tolerance of 0.8V DC, and a current of 0.3 mA. The power source 116 may be selectively electrically coupled to the active and counter electrode assemblies 112a, 14 via a control circuit 92 (discussed below), for example, via carbon fiber ribbons 94a, 94b. The iontophoresis device 5 may include a controller 96 and a regulating circuit 98 (discussed below) formed from discrete and/or integrated circuit elements to control and/or monitor operation, and/or regulate the voltage, current and/or power delivered to the electrode assemblies 112a, 114. For example, the iontophoresis device 5 may include a diode to provide a constant current to the electrode elements 120, 140.

As suggested above, the active agent 124 may take the form of a cationic or an anionic drug or other therapeutic agent. Consequently, the poles or terminals of the power source 116 may be reversed. Likewise, the selectivity of the outermost ion selective membranes 122, 142 and inner ion selective membranes 134, 54 may be reversed.

The control circuit 92 includes the controller 96 and regulating circuit 98, which may be mounted or carried by a circuit board, such as flexible circuit board 100. The flexible circuit board 100 may comprise one or more insulative layers, and may optionally comprise one or more conductive layers interlaced with the insulative layers. The circuit board 100 may form one or more vias (not shown), to make electrical couplings between the surfaces of the circuit board and/or between various ones of the conductive layers.

The control circuit 92 may also include one or more current sensors 102a-102d (collectively 102), positioned and configured to sense or measure current through one or more reservoirs, membranes or other structures. The control circuit 92 may also include one or more voltage sensors 104a-104c (collectively 104), positioned and configured to sense or measure voltage across one or more reservoirs, membranes or other structures. The current and voltage sensors 102, 104 provide signals indicative of the current i₁-iₙ, and signals indicative of the voltage v₁-vₘ, respectively, to the controller 96.

The control circuit 92 may also include an off-chip oscillator 106 that provides a frequency signal to the controller 96 to form a clock signal. Alternatively, the controller 92 may employ an on-chip oscillator.

The control circuit may further include an end user interface 97 that may be operated by the end user of the iontophoresis device 4 to make a request, for example to request a change in active agent dosage such as, for example, an increase or decrease in dosage.

The controller 92 may employ the signals indicative of the current i₁-iₙ, and signals indicative of the voltage v₁-vₘ, as well as the frequency signals to analyze operation of the device, and to produce additional performance information, as discussed in more detail below.

The radio 108 may include a transmitter 108a and/or receiver 108b, which may be formed as a transceiver, which may be coupled to one or more active radiating antenna elements, for example dipole antenna 110a. The controller 92 is communicatively coupled to receive and/or provide information from and/or to the radio 108. Thus, the controller 92 may cause the transmitter 108a to transmit operational parameters and/or requests (e.g., request for adjustment of the active agent delivery) from the iontophoresis device 4. Likewise, the controller 92 may receive commands to adjust at least one of the operational parameters, from the iontophoresis device environment server 16 and/or the iontophoresis interface system 2 via the receiver 108b.

The controller 96 may use the operational parameters and/or other performance information that it generates or collects, as well as commands received from the iontophoresis interface system 2 and/or the iontophoresis device environment server 16 to adjust the at least one operational parameter, to modify an operation of the iontophoresis device 4, for example the active agent delivery regime. For example, the controller 96 may implement a new or updated active agent delivery regime based on a command to adjust at least one operational parameter by providing appropriate control signals to the regulating circuit 98 to implement the new or revised regime. The regulating circuit 98 may take the form of a voltage control regulator and/or current control regulator that controls the delivery of active agent by controlling voltage applied across, or current applied to, the electrode elements 124, 168.

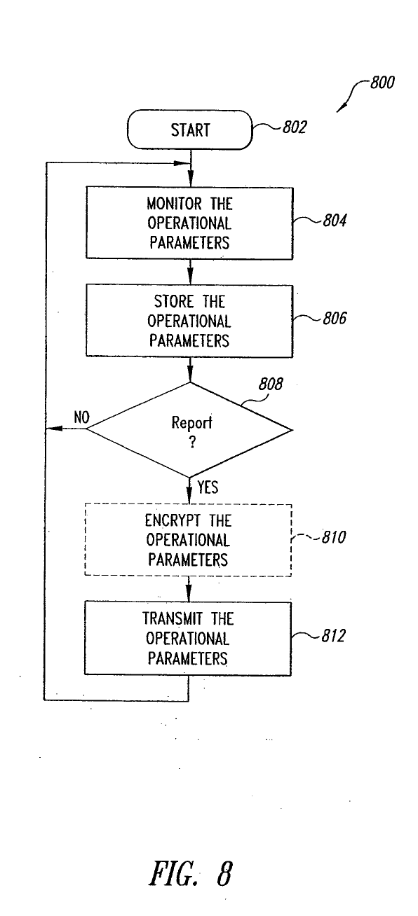

FIG. 8 is a high level flow diagram of a method 800 of operating the iontophoresis device 4 to monitor and report operational parameters and/or performance information, according to one illustrated embodiment. The method 800 may be implemented by the controller 96, as either software or firmware instructions, or as hardwired logic.

The method 800 starts at 802, for example in response to an activation of the iontophoresis device 4. As discussed in more detail below, at 804 the controller 96 monitors the operational parameters and/or performance of the iontophoresis device 4.

Optionally at 806, the controller 96 stores operational parameters and/or other performance information. The storage may be to one or more registers of the controller 96, or memory structures (not shown) associated with the controller 96, such as random access memory (RAM).

Optionally at 808, the controller 96 determines whether or not to wirelessly report the operational parameters and/or other performance information. As discussed in more detail below, reporting may be in response to an inquiry or command, for example, from the iontophoresis interface system, and/or in response to the expiration of a period or time. In particular, the controller 96 may, for example, check a report flag that may be set via another process or thread. If the report flag is set to a logical value corresponding to yes, the method 800 passes control to 810 or 812. Otherwise the method 800 passes control back to 804.

Optionally at 810, the controller 96 encrypts the desired operational parameters and/or other performance information. Encryption advantageously reduces the ability of third parties to mischievously interfere with the provisional of medical services. Encryption also advantageously protects personal medical information, which may be a legal requirement in some jurisdictions. The controller 96 may employ any of a variety of standard encryption algorithms. For example, the controller 96 may employ an encryption algorithm based on public/private key pairs. The public key may belong to a specific iontophoresis interface system 2 to which the information will be sent, or may be generic to a few or a large number of remote iontophoresis devices 4.

At 812, the controller 96 transmits the operational parameters and/or other performance information. The controller 96 may forward appropriate signals to the transmitter 108a of the radio 108 to cause transmission of the operational parameters and/or other performance information. The iontophoresis device 4 may include additional structures, such as a digital-to-analog converter between the controller 96 and transmitter 108a. Alternatively, the radio 108 may implement a digital-to-analog conversion, if necessary or convenient.

The transmission may be a broadcast, or alternatively a pointcast. The transmission can employ any known or later developed protocol, including: time division multiple access (TDMA), frequency division multiple access (FDMA), code division multiple access (CDMA), spread spectrum, and/or BLUETOOTH®.

After transmission, control returns to 804.

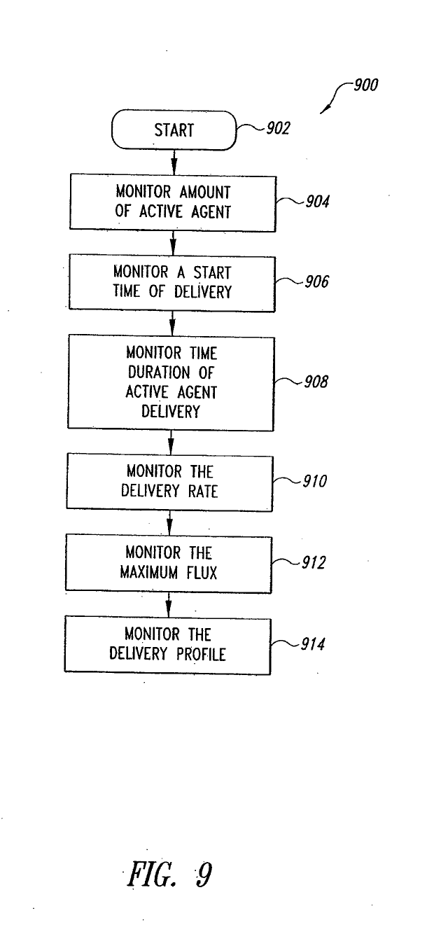

FIG. 9 is a low level flow diagram of a method 900 of monitoring operational parameter information according to one illustrated embodiment, the method useful in the method of FIG. 8. The method 900 may be implemented by the controller 96, as either software or firmware instructions, or as hardwired logic.

The method 900 starts at 902. For example, the method 900 may start in response to an activation of the iontophoresis device 4, and may run in parallel with method 800, for example as a separate process or thread. Activation may be the closing of a switch, or simply the application of the iontophoresis device 4 to the biological interface 118 that completes the circuit. Alternatively, the method 900 may start in response to a call request or inquiry from the controller 96, for example, at 804 of method 800 (FIG. 8).

At 904, the controller 96 monitors a total amount of active agent delivered. For example, the controller 96 may monitor a current through a reservoir, membrane or other structure, and/or may monitor a voltage across a reservoir, membrane or other structure to determine the total amount of active agent delivered. For instance, the controller 96 may monitor the amount of current drawn over an entire period of time during which the active agent is delivered, and determine the amount of active agent delivery based on a defined relationship between current and rate of active agent delivery, based on the knowledge of the total time of delivery. Such may be refined using empirically derived relationships.

At 906, the controller 96 monitors a time at which a delivery of the active agent starts. For example, the controller 96 may start a timer or clock when current beings to flow, for example in response to activation of a switch or simply the completion of the circuit by the placement of the iontophoresis device 4 on the biological interface 18 (FIG. 7).

At 908, the controller 96 monitors a duration during which the active agent is delivered. For example, the controller 96 may stop a timer or clock when current stops flowing, for example in response to deactivation of a switch or simply the opening of the circuit path between the electrode assemblies 12, 14 by the removal of the iontophoresis device 4 from the biological interface 18 (FIG. 7).

At 910, the controller 96 monitors a rate at which the active agent is delivered. For example, the controller 96 may monitor a current through a reservoir, membrane or other structure, and/or may monitor a voltage across a reservoir, membrane or other structure to determine the rate at which the active agent is delivered. For instance, the controller 96 may monitor an instantaneous rate based on a relationship between current and rate of delivery and a knowledge of the instantaneous current. Also for instance, the controller 96 may monitor an average rate by cumulating or integrated the instantaneous rates.

At 912, the controller 96 monitors a maximum flux at which the active agent is delivered. For example, the controller 96 may monitor a current through a reservoir, membrane or other structure, and/or may monitor a voltage across a reservoir, membrane or other structure to determine the maximum flux at which the active agent is delivered. For instance, the controller 96 may monitor the maximum current draw. The controller 96 may determine the maximum flux based on a relationship between current and rate of delivery, and a knowledge of the maximum current draw.

At 914, the controller 96 monitors a delivery profile at which the active agent is delivered. For example, the controller 96 may monitor a current through a reservoir, membrane or other structure, and/or may monitor a voltage across a reservoir, membrane or other structure to determine the total amount of active agent delivered. For instance, the controller 96 may monitor the current over time, determining the delivery profile based at least in part on a relationship between current and rate of delivery, and a knowledge of the instantaneous current through the active agent delivery. Such may be refined using empirically derived relationships, for example, a relationship between rate of delivery and voltage, a relationship between rate of delivery and impedance where impedance is either monitored or determined from another monitored parameter (e.g., current or voltage). The method 900 may include additional acts, may omit some of the above-described acts and/or may perform acts in a different order than set out in the flow diagram.

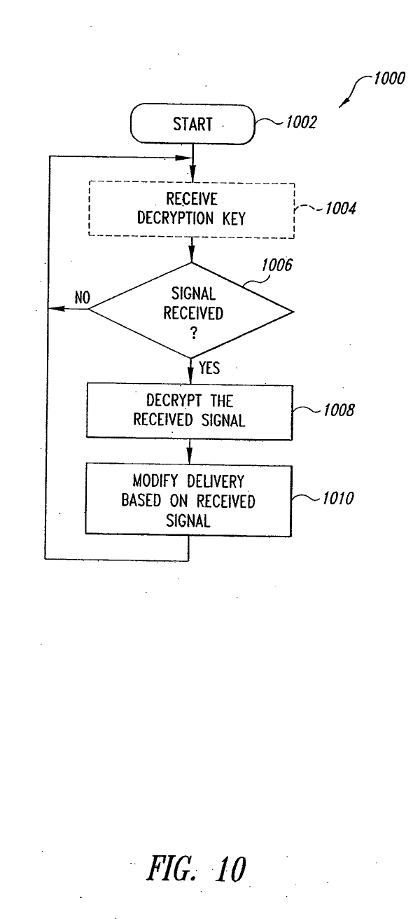

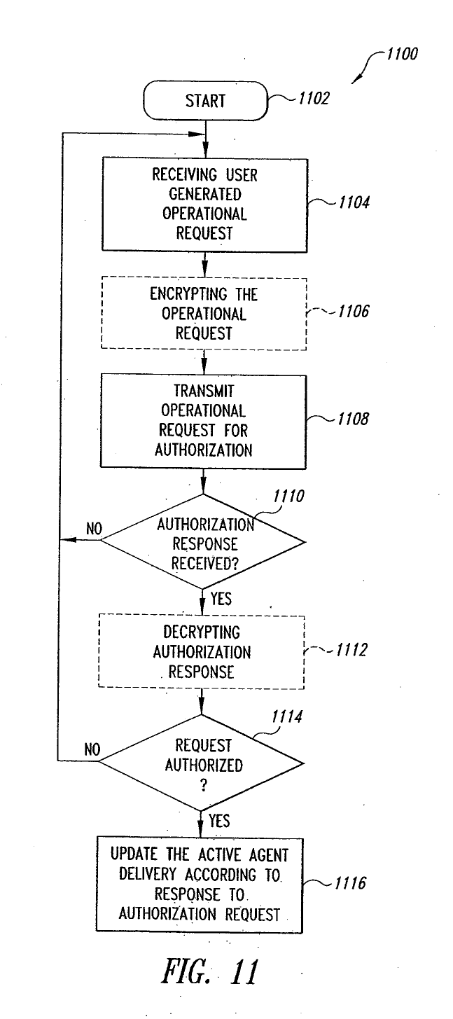

FIG. 10 is a high level flow diagram of a method 1000 of operating the iontophoresis device 4 to receive requests for adjusting at least one operational parameter and modifying the active agent delivery in response thereto, according to one illustrated embodiment. The method 1000 may be implemented by the controller 96, as either software or firmware instructions, or as hardwired logic.

The method 1000 starts at 1002, for example in response to an activation of the iontophoresis device 4.

Optionally, at 1004 the controller 96 receives a decryption key from the iontophoresis interface system 2 and/or iontophoresis device environment server 16. This permits the controller 96 to decrypt requests for the adjustment of at least one operational parameter to be sent to the iontophoresis delivery device 4.

At 1006, the controller 96 determines whether a signal is received. The controller 96 may use any of a variety of known or later developed methods and circuits for detecting the receipt of a transmission. If a signal is not received, a wait loop is executed, with control passing back to 1004. If a signal is received, control passes to 1008.

Optionally at 1008, the controller 96 decrypts and/or decodes the received signal. For example, the controller 96 may decrypt the signal using the decryption key previously provided by the iontophoresis interface system 2 to the iontophoresis device 4, or using a generic decryption key common to a number of iontophoresis devices 4. The controller 96 may decode the information using any suitable decoding methods or structures currently known or later developed. Such methods and/or structures are commonly known in the telecommunications industry (TDMA, FDMA, CDMA), and may, for example, include up and/or down mixers.

At 1010, the controller 96 modifies the active agent delivery regime based on the received command for adjusting at least one operational parameter, by providing appropriate control signals to the regulating circuit to implement the revised regime. Modifying the active agent delivery regime based on the at least one operational parameter is described in more detail below.

The method 1000 passes control to 1004 and waits for receipt of further command signals. The method 1000 may include additional acts, may omit some of the above-described acts and/or may perform acts in a different order than set out in the flow diagram.

FIG. 11 is a high level flow diagram of a method 1100 of operating the iontophoresis device 4 to provide authorizing requests to the iontophoresis interface system 2 and/or iontophoresis device environment server 16 based on user generated operational requests, and responding to commands provided from the iontophoresis interface system 2 and/or iontophoresis device environment server 16 in response to the authorizing requests, according to one embodiment.

The method 1100 starts at 1102, for example in response to the activation of the iontophoresis device 4.

At 1104, the controller 96 receives user generated operational requests via the end user interface 97 operable by the end user of the iontophoresis device 4. The end user interface may, for example, comprise buttons or icons operable to request a change in device operation, for example a change in the dosage. The iontophoresis device 4 may automatically implant the request if the requested change is within a predefined limit or threshold, as set by the health care professional, or may send an authorization request to the iontophoresis interface system 2 when the request is outside the predefined limit.

Optionally at 1106, the controller 96 encrypts the operational requests that require authorization. The encryption mechanism is as describe above.

At 1108, the controller 96 transmits the operational requests that require authorization. The controller 96 may forward appropriate signals to the transmitter 108a of the radio 108 to cause transmission of the operational requests. The user generated operational requests may be transmitted to the iontophoresis interface system 2 for authorization, directly or via the iontophoresis device environment server 16.

At 1110, the controller 96 determines if an authorization response signal has been received. The controller 96 may use any of a variety of known or later developed methods and circuits for detecting the receipt of a transmission. If a signal is not received, a wait loop is executed, with control passing back to 1104. If a signal is received, control passes to 1112 or 1114.

Optionally at 1112, the controller 96 decrypts the authorization response. The decryption and/or decoding is as described above in FIG. 10.

At 1114, the controller 96 determines if the request for adjusting at least one operational parameter is rejected. If the request was rejected, control passes back to 1104. If the request was authorized, control passes to 1116.

At 1116, the controller 96 updates the active agent delivery regime based on the response to the request for authorization caused by the end user generated request for adjustment of the at least one operational parameter. The controller 96 accomplishes such by providing appropriate signals to the regulating circuit to implement the revised regime. The adjustment of the active agent delivery regime based on at least one operational parameter is described in more detail below.

The method 1100 may include additional acts, may omit some of the above-described acts and/or may perform acts in a different order than set out in the flow diagram.

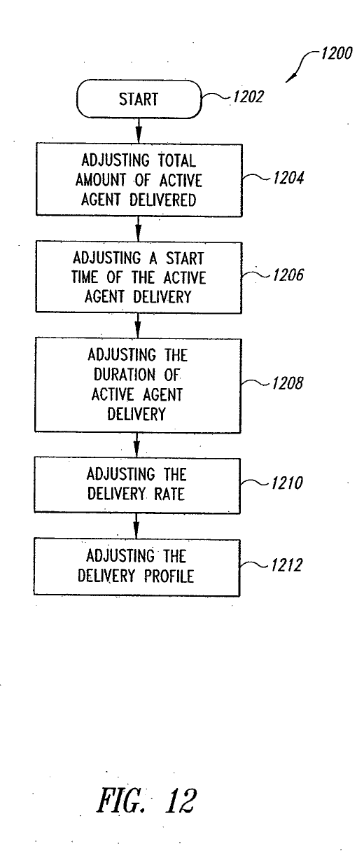

FIG. 12 is a low level flow diagram of a method 1200 of adjusting the active agent delivery regime based on the at least one operational parameter information according to one illustrated embodiment, the method useful in the methods of FIGS. 10 and 11. The method 1200 may be implemented by the controller 96, as either software or firmware instructions, or as hardwired logic. Alternatively, the method 1200 may start in response to a call from the controller 96, for example, at 1010 or 1116 of methods 1000 and 1100 respectively (FIGS. 10 and 11).

The method 1200 starts at 1202. For example, the method 1200, may start in response to commands for adjusting a desired operational parameter to modify the active agent delivery, and may run in parallel with methods 1000 and 1100, for example as a separate process or thread. The commands may be received based on the end user generated operational request (as in method 1100) or on the commands provided from the iontophoresis interface system 2 (as in method 1000).

At 1204, the controller 96 adjusts a total amount of active agent delivered. For example, the controller 96 may adjust a current through a reservoir, membrane or other structure, and/or may adjust a voltage across a reservoir, membrane or other structure to modify the total amount of active agent delivered. For instance, the controller 96 may modify the amount of current drawn over an entire period of time during which the active agent is delivered, and determine the amount of active agent delivery based on a defined relationship between current and rate of active agent delivery, based on the knowledge of the total time of delivery. Such may be refined using empirically derived relationships.

At 1206, the controller 96 adjusts a time at which a delivery of the active agent starts. For example, the controller 96 may start a timer or clock when current beings to flow, for example in response to activation of a switch.

At 1208, the controller 96 modifies a duration during which the active agent is delivered. For example, the controller 96 may stop a timer or clock when current stops flowing, for example in response to deactivation of a switch.

At 1210, the controller 96 modifies a rate at which the active agent is delivered. For example, the controller 96 may modify a current through a reservoir, membrane or other structure, and/or may modify a voltage across a reservoir, membrane or other structure to modify the rate at which the active agent is delivered. For instance, the controller 96 may modify an instantaneous rate based on a relationship between current and rate of delivery and a knowledge of the instantaneous current. Also for instance, the controller 96 may modify an average rate by cumulating or integrating the modified instantaneous rates.

At 1212, the controller 96 modifies a delivery profile at which the active agent is delivered. For example, the controller 96 may modify a current through a reservoir, membrane or other structure, and/or may modify a voltage across a reservoir, membrane or other structure to modify the total amount of active agent delivered. For instance, the controller 96 may modify the current over time, determining the delivery profile based at least in part on a relationship between the current and rate of delivery, and a knowledge of the instantaneous current through the active agent delivery. Such may be refined using empirically derived relationships, for example, a relationship between rate of delivery and voltage, a relationship between rate of delivery and impedance where impedance is modified by adjusting another monitored parameter (e.g., current or voltage).

The method 1200 may include additional acts, may omit some of the above-described acts and/or may perform acts in a different order than set out in the flow diagram.

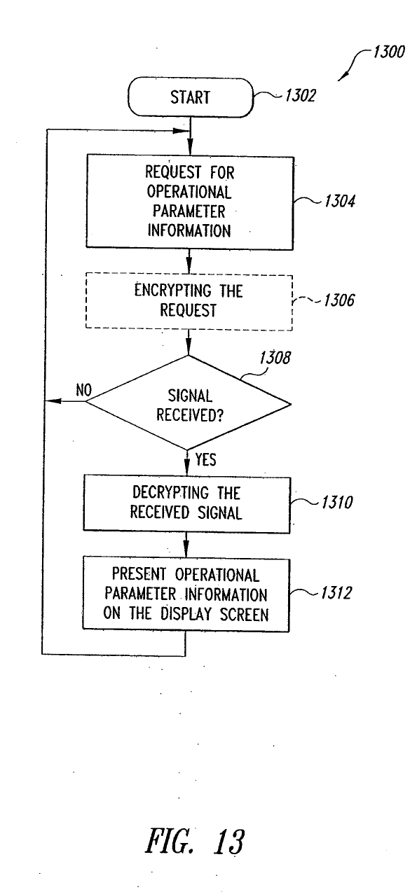

FIG. 13 shows a high level flow diagram of a method 1300 of operating the iontophoresis interface system 2 to communicate commands for monitoring operational parameter information to the respective iontophoresis devices 4, according to one illustrated embodiment. The method 1300 may be implemented via the iontophoresis user interface 46, 50.

The method 1300 starts at 1302, for example in response to the activation of the iontophoresis user interface system 2.

At 1304, the health care professional requests for specific operational parameter information from the desired iontophoresis devices 4, wherein the request is initiated via the iontophoresis user interface system 2 (as described in detail above).

Optionally at 1306, the request for operational parameter information is encrypted, the encryption is described in detail above (see FIG. 9).

At 1308, the iontophoresis interface system 2 determines whether a signal is received. The iontophoresis interface system 2 may use any of a variety of known or later developed methods and circuits for detecting the receipt of a transmission. If a signal is not received, a wait loop is executed, with control passing back to 1304. If a signal is received, control passes to 1310.

At 1310, the iontophoresis interface system 2 decrypts and/or decodes the received signal. For example, the iontophoresis interface system 2 may decrypt the signal using a decryption key previously provided by the iontophoresis device 4 to the iontophoresis interface system 2, or using a generic decryption key. The iontophoresis interface system 2 may decode the information using any suitable decoding methods or structures currently known or later developed. Such methods and/or structures are commonly known in the telecommunications industry (TDMA, FDMA, CDMA), and may, for example, include up and/or down mixers.

At 1312, the operational parameter information for the desired iontophoresis device 4 is provided on the display screen 82, 83 presented by the iontophoresis user interface system 2 (as described in detail above).

The method 1300 passes control to 1304 and waits for receipt of further user generated requests for operational parameter information.

The method 1300 may include additional acts, may omit some of the above-described acts and/or may perform acts in a different order than set out in the flow diagram.

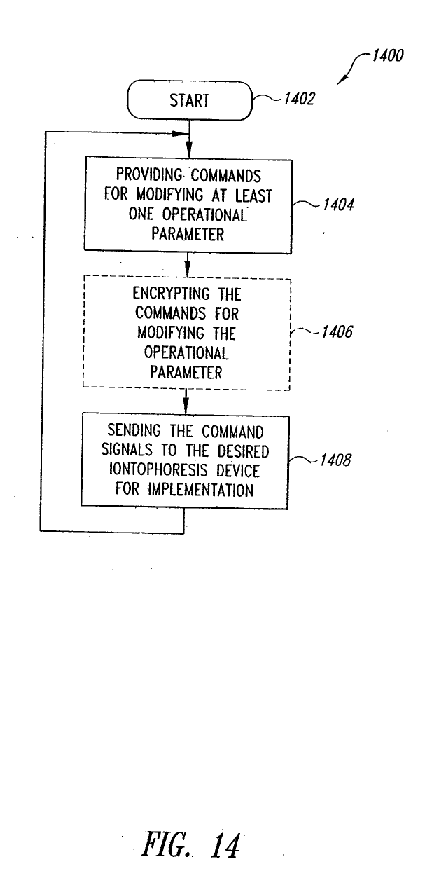

FIG. 14 shows a high level flow diagram of a method 1400 of operating the iontophoresis interface system 2 to communicate commands for modifying operational parameter information to the respective iontophoresis devices 4, according to one illustrated embodiment. The method 1400 may be implemented via the iontophoresis interface 46, 50

The method 1400 starts at 1402, for example in response to the activation of the iontophoresis user interface system 2.

At 1404, the health care professional provides commands for modification of at least one operational parameter to adjust the active agent delivery regime of the desired iontophoresis devices 4, wherein the commands for modification are initiated via the iontophoresis user interface 46, 50 (as described in detail above).

Optionally at 1406, the commands for modifying at least one operational parameter of the iontophoresis device 4 are encrypted, the encryption is described in detail above (see FIG. 9).

At 1408, the command signals are sent to the desired iontophoresis devices 4 for implementation via the controller and regulator.

The method 1400 passes control to 1404 and waits for receipt of further user generated requests for modifying at least one operational parameter to adjust the active agent delivery regime.

The method 1400 may include additional acts, may omit some of the above-described acts and/or may perform acts in a different order than set out in the flow diagram.

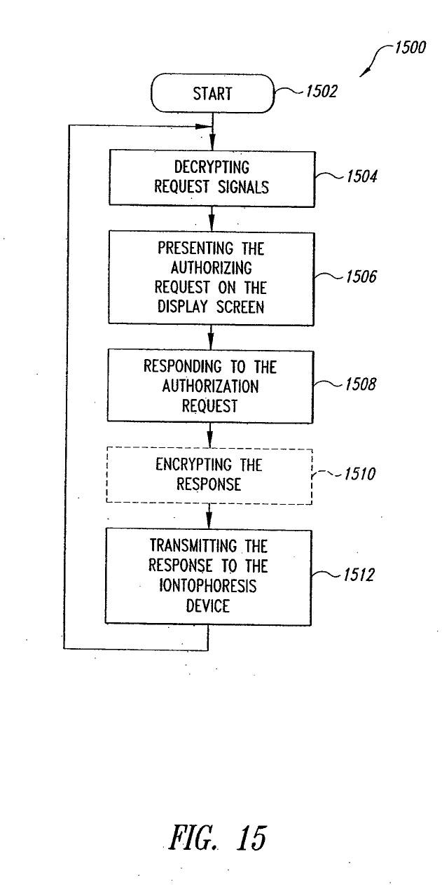

FIG. 15 shows a high level flow diagram of a method 1500 of operating the iontophoresis interface system 2 to communicate commands for modifying operational parameter information in response to authorization requests received from respective iontophoresis devices 4, according to one illustrated embodiment. The method 1500 may be implemented via the iontophoresis interface 46, 50.

The method 15 starts at 1502, for example when at least one iontophoresis device 4 sends a request to authorize an adjustment of at least one operational parameter, wherein the authorization is granted either automatically through the iontophoresis user interface system 2 or manually through the iontophoresis user interface 46, 50 (as described in detail above).

At 1504, the iontophoresis interface system 2 decrypts and/or decodes the received request signal. For example, the iontophoresis interface system 2 may decrypt the request signal using a decryption key previously provided by the iontophoresis device 4 to the iontophoresis interface system 2, or using a generic decryption key. The iontophoresis interface system 2 may decode the information using any suitable decoding methods or structures currently known or later developed. Such methods and/or structures are commonly known in the telecommunications industry (TDMA, FDMA, CDMA), and may, for example, include up and/or down mixers.

At 1506, the authorization request for the adjustment of the operational parameter is presented on the display 83 of the iontophoresis user interface system 2 (as described in detail above).

At 1508, the health care professional responds to the authorization request for modifying the operational parameter via the iontophoresis user interface 46, 50 presented by the iontophoresis user interface system 2 (as described in detail above).

Optionally at 1510, the response to the authorization request for modifying the at least one operational parameter is encrypted, the encryption is described in detail above (see FIG. 9).

At 1512, the response to the authorization request for modifying the at least one operational parameter is transmitted to the iontophoresis device 4 which initiated the response.

The method 1500 passes control to 1504 and waits for receipt of further authorization requests from the iontophoresis device 4.

The method 1500 may include additional acts, may omit some of the above-described acts and/or may perform acts in a different order than set out in the flow diagram.

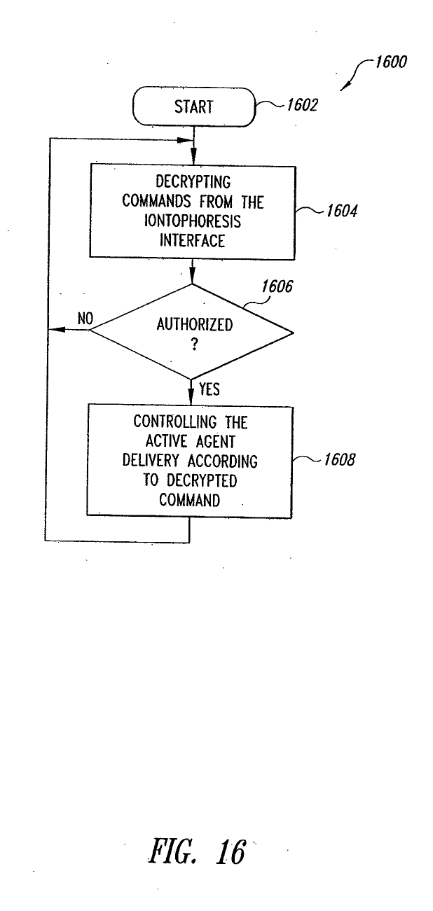

FIG. 16 shows a high level flow diagram of a method 1600 of operating an iontophoresis system 1 to at least partially control the active agent delivery of the iontophoresis device 4, according to one illustrated embodiment.

The method 16 starts at 1602, for example by providing communications between a plurality of iontophoresis devices 4 and at least one iontophoresis interface system 2 remotely located from the iontophoresis devices 4.

At 1604, the iontophoresis device 4 decrypts commands provided from the iontophoresis interface system 2. Decrypting is described in detail above.

At 1606, the iontophoresis device 4 determines whether the commands provided from the iontophoresis interface system 2 are authentic. Commands that cannot be authenticated are at least initially rejected. If the command is rejected, control passes back to 1604. If the command is determined to be authentic, control passes to 1608. Authentication may be done in any known method of authentication, including via public/private keys.

At 1608, the iontophoresis device 4 at least partially controls or adjusts the delivery of the active agent to the biological interface 118 from the at least one iontophoresis device 4 in response to commands provided from the iontophoresis interface system 2 via the transmission connector 10. Commands provided from the iontophoresis interface system 2 may be in response to the user generated requests for authorization. The controlling or adjusting of the active agent delivery is based on at least one operational aspect as described in more detail above.

The method 1600 passes control to 1604 and waits for communication of commands between the plurality of iontophoresis devices 4 and the iontophoresis interface system 2.

The method 1600 may include additional acts, may omit some of the above-described acts and/or may perform acts in a different order than set out in the flow diagram.

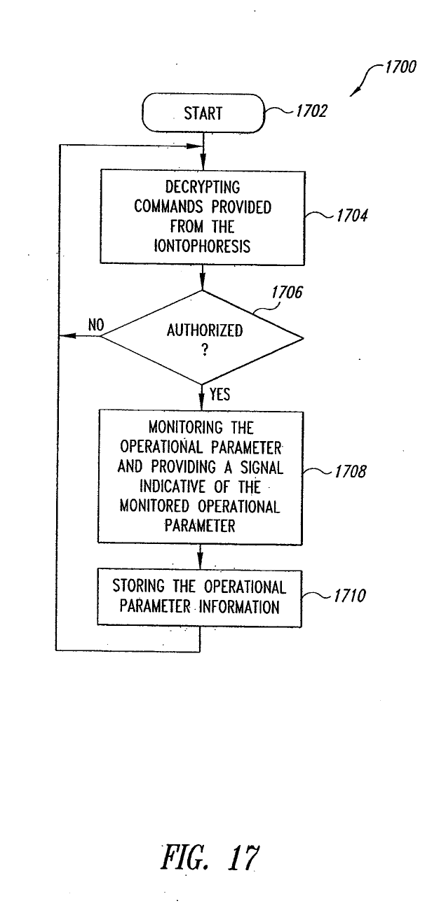

FIG. 17 shows a high level flow diagram of a method 1700 of operating an iontophoresis system 1 to monitor the active agent delivery of the iontophoresis device 4, according to one illustrated embodiment.

The method 17 starts at 1702, for example by providing communications between a plurality of iontophoresis devices 4 and at least one iontophoresis interface system 2 remotely located from the iontophoresis devices 4.

At 1704, the iontophoresis device 4 decrypts the commands provided from the iontophoresis interface system 2. Decryption is described in detail above.

At 1706, the iontophoresis device 4 determines whether the commands provided from the iontophoresis interface system 2 are authentic. The iontophoresis device 4 at least initially rejects any commands that are determined not to be authenticated. If the command is rejected, control passes back to 1704. If the command is determined to be authentic, control passes to 1708.

At 1708, the iontophoresis device 4 monitors at least one operational aspect of the iontophoresis device 4 according to the commands provided from the iontophoresis interface system 2. The iontophoresis device 4 provides signals indicative of the monitored operational aspect to the iontophoresis interface system 2. Monitoring the operational parameters and/or performance of the iontophoresis device 4 is described above.

Optionally at 1710, the iontophoresis device 4 at least temporarily stores operational condition information at the iontophoresis interface system 2. The operational information may be indicative of at least one operating parameter for one or more of the iontophoresis devices 4.

The method 1700 passes control to 1704 and waits for communication to be provided between the plurality of iontophoresis devices 4 and at least one iontophoresis interface system 2.

The above description of illustrated embodiments, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to wireless or hard wired communication systems, not necessarily the exemplary iontophoresis system generally described above.

For instance, the foregoing detailed description has set forth various embodiments of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

In addition, those skilled in the art will appreciate that the mechanisms of taught herein are capable of being distributed as a program product in a variety of forms, and that an illustrative embodiment applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to:

Japanese patent application Serial No. H03-86002, filed Mar. 27, 1991, having Japanese Publication No. H04-297277, issued on Mar. 3, 2000 as Japanese Patent No. 3040517;

Japanese patent application Serial No. 11-033076, filed Feb. 10, 1999, having Japanese Publication No. 2000-229128;

Japanese patent application Serial No. 11-033765, filed Feb. 12, 1999, having Japanese Publication No. 2000-229129;

Japanese patent application Serial No. 11-041415, filed Feb. 19, 1999, having Japanese Publication No. 2000-237326;

Japanese patent application Serial No. 11-041416, filed Feb. 19, 1999, having Japanese Publication No. 2000-237327;

Japanese patent application Serial No. 11-042752, filed Feb. 22, 1999, having Japanese Publication No. 2000-237328;

Japanese patent application Serial No. 11-042753, filed Feb. 22, 1999, having Japanese Publication No. 2000-237329;

Japanese patent application Serial No. 11-099008, filed Apr. 6, 1999, having Japanese Publication No. 2000-288098;

Japanese patent application Serial No. 11-099009, filed Apr. 6, 1999, having Japanese Publication No. 2000-288097;

PCT patent application WO 2002JP4696, filed May 15, 2002, having PCT Publication No W003037425;

U.S. patent application Ser. No. 10/488,970, filed Mar. 9, 2004;

Japanese patent application 2004/317317, filed Oct. 29, 2004;

U.S. provisional patent application Ser. No. 60/627,952, filed Nov. 16, 2004;

Japanese patent application Serial No. 2004-347814, filed Nov. 30, 2004;

Japanese patent application Serial No. 2004-357313, filed Dec. 9, 2004;

Japanese patent application Serial No. 2005-027748, filed Feb. 3, 2005; and

Japanese patent application Serial No. 2005-081220, filed Mar. 22, 2005.

Aspects of the invention can be modified, if necessary, to employ systems, circuits and concepts of the various patents, applications and publications to provide yet further embodiments of the invention.

These and other changes can be made to the invention in light of the above-detailed description. In general, in the following claims, the terms used should not be construed to limit the invention to the specific embodiments disclosed in the specification and the claims, but should be construed to include all medical devices and/or communication systems that operated in accordance with the claims. Accordingly, the invention is not limited by the disclosure, but instead its scope is to be determined entirely by the following claims.

I/We claim:

1. An iontophoresis device, comprising:
an active agent reservoir that stores a quantity of an active agent;
an active electrode element operable to apply an electrical potential of a first polarity to deliver at least a portion of the active agent to a biological interface from the iontophoresis device; and
a communications port providing a communications path between the iontophoresis device and a remote device wherein the remote device provides commands to the iontophoresis device that are encrypted.

2. The iontophoresis device of claim 1, further comprising:
a controller operable to decrypt the commands provided from the remote device via the communications port and to at least partially control the delivery of the active agent to the biological interface from the iontophoresis device based at least partially on the decrypted commands.

3. The iontophoresis device of claim 2 wherein the controller is configured to determine whether the commands provided from the remote device via the communications port are authorized, and to at least initially reject any commands that are determined not to be authorized.

4. The iontophoresis device of claim 2, further comprising:
at least one ion selective membrane position between the active agent reservoir and an exterior of the iontophoresis device to at least partially control the delivery of the active agent to the biological interface from the iontophoresis device.

5. The iontophoresis device of claim 2, further comprising:
at least one ion selective membrane position between the active electrode element and the active agent reservoir.

6. The iontophoresis device of claim 1, further comprising:
a user interface operable by a user to receive user generated operational requests; and
a controller operable to decrypt the commands provided from the remote device via the communications port in response to authorizing requests provided from the controller to the remote device based on the user generated operational requests and to at least partially control the delivery of the active agent to the biological interface from the iontophoresis device based at least partially on the decrypted commands.

7. The iontophoresis device of claim 6 wherein the controller is configured to encrypt authorizing requests to the remote device via the communications port.

8. The iontophoresis device of claim 6 wherein the controller is configured to determine whether the commands provided from the remote device via the communications port are authorized, and to at least initially reject any commands that are determined not to be authorized.

9. An iontophoresis interface system, comprising:
a user interface operable by a user to produce user generated requests and to provide information to the user;
a communications port providing a communications path between the iontophoresis interface system and at least one iontophoresis device remotely located with respect to the iontophoresis interface system; and
a processor coupled to control communications with the at least one iontophoresis device via the communications port based at least in part on the user generated requests.

10. The iontophoresis interface system of claim 9 wherein in user interface is operable to produce user generated requests for information indicative of an operational condition of the at least one iontophoresis device.

11. The iontophoresis interface system of claim 9 wherein in user interface is operable to produce user generated requests for information indicative of a rate of transfer of an active agent by the at least one iontophoresis device, and to provide information to the user indicative of the rate of transfer of an active agent by the at least one iontophoresis device.

12. The iontophoresis interface system of claim 9 wherein in user interface is operable to produce user generated requests for information indicative of an amount of an active agent transferred by the at least one iontophoresis device, and to provide information to the user indicative of the amount of an active agent transferred by the at least one iontophoresis device.

13. The iontophoresis interface system of claim 9 wherein in user interface is operable to produce user generated requests for information indicative of a current flow through at least a portion of the at least one iontophoresis device, and to provide information to the user indicative of the current flow through at least a portion of the at least one iontophoresis device.

14. The iontophoresis interface system of claim 9 wherein in user interface is operable to produce user generated requests for information indicative of a voltage across at least a portion of the at least one iontophoresis device, and to provide information to the user indicative of the voltage across at least a portion of the at least one iontophoresis device.

15. The iontophoresis interface system of claim 9 wherein in user interface is operable to produce user generated requests for information indicative of a time during which the iontophoresis device has been at least one of active or inactive, and to provide information to the user indicative of the time during which the iontophoresis device has been at least one of active or inactive.

16. The iontophoresis interface system of claim 9 wherein in user interface is operable to produce user generated requests for information indicative of a delivery profile of the active agent over a period of time, and to provide information to the user indicative of the delivery profile of the active agent over the period of time.

17. The iontophoresis interface system of claim 9 wherein the communications port comprises at least one antenna element.

18. The iontophoresis interface system of claim 9 wherein the communications port comprises at least one communications connector.

19. The iontophoresis interface system of claim 9 wherein the communications path is at least one of a wired network connection or wireless network connection.

20. The iontophoresis interface system of claim 9 wherein the processor is operable to provide commands to the at least one iontophoresis device from the iontophoresis interface system via the communications port based at least in part on the user generated requests.

21. The iontophoresis interface system of claim 20 wherein at least some of the commands provided by the iontophoresis interface system to the at least one iontophoresis device are indicative of a delivery rate for delivering active agent to a biological interface from the iontophoresis device.

22. The iontophoresis interface system of claim 20 wherein at least some of the commands provided by the iontophoresis interface system to the at least one iontophoresis device are indicative of an amount of an active agent to deliver to a biological interface from the iontophoresis device.

23. The iontophoresis interface system of claim 20 wherein at least some of the commands provided by the iontophoresis interface system to the at least one iontophoresis device are indicative of a current flow through at least a portion of the iontophoresis device.

24. The iontophoresis interface system of claim 20 wherein at least some of the commands provided by the iontophoresis interface system to the at least one iontophoresis device are indicative of a voltage across at least a portion of the iontophoresis device.

25. The iontophoresis interface system of claim 20 wherein at least some of the commands provided by the iontophoresis interface system to the at least one iontophoresis device are indicative of a time during which the iontophoresis device is to actively deliver an active agent to a biological interface.

26. The iontophoresis interface system of claim 20 wherein at least some of the commands provided by the iontophoresis interface system to the at least one iontophoresis device are indicative of a delivery profile for delivering an active agent from the iontophoresis device to a biological interface.

27. The iontophoresis interface system of claim 20 wherein the communications port comprises at least one antenna element.

28. The iontophoresis interface system of claim 20 wherein the communications port comprises at least one communications connector.

29. The iontophoresis interface system of claim 20 wherein the communications path is at least one of a wired network connection or wireless network connection.

30. An iontophoresis system, comprising:
at least a first iontophoresis device operable to deliver an active agent to a biological interface, the first iontophoresis device comprising an active agent reservoir that stores a quantity of the active agent and an active electrode element operable to apply an electrical potential of a first polarity to deliver at least a portion of the active agent to the biological interface from the iontophoresis device; and
an iontophoresis interface system remotely located with respect to the first iontophoresis device, the iontophoresis interface system comprising a user interface operable by a user to produce user generated requests and to provide information to the user, a communications port providing a communications path between the iontophoresis interface system and the at least one iontophoresis device, and a processor coupled to control communications with the at least one iontophoresis device via the communications port based at least in part on the user generated requests.

31. The iontophoresis system of claim 30 wherein the first iontophoresis device further comprises a controller operable to at least partially control the delivery of the active agent to the biological interface from the iontophoresis device, wherein the controller is responsive to commands provided from the iontophoresis interface system via the communications port.

32. The iontophoresis system of claim 31 wherein the controller decrypts the commands provided from the iontophoresis interface system via the communications port.

33. The iontophoresis system of claim 31 wherein the controller is operable to determine whether the commands provided from the iontophoresis interface system via the communications port are authorized, and to at least initially reject any commands that are determined not to be authorized.

34. The iontophoresis system of claim 30 wherein the iontophoresis device comprises a user interface operable by a user to receive user generated operational requests and a controller operable to at least partially control the delivery of the active agent to the biological interface from the iontophoresis device, and wherein the controller is operable to provide authorizing requests to the iontophoresis interface system based on the user generated operational requests, and is responsive to commands provided from the iontophoresis interface system via the communications port in response to the authorizing requests.

35. The iontophoresis system of claim 34 wherein the controller encrypts authorizing requests to the iontophoresis interface system and decrypts the commands provided from the iontophoresis interface system via the communications port.

36. The iontophoresis system of claim 34 wherein the controller is operable to determine whether the commands provided from the iontophoresis interface system via the communications port are authorized, and to at least initially reject any commands that are determined not to be authorized.

37. The iontophoresis system of claim 30 wherein the iontophoresis device further comprises a monitoring subsystem operable to monitor at least one operational aspect of the iontophoresis device and coupled to the communications port to provide signals indicative of the monitored operational aspect to the iontophoresis interface system via the communications path.

38. The iontophoresis system of claim 37 wherein the monitoring subsystem is implemented in a controller operable to at least partially control the delivery of the active agent to the biological interface from the iontophoresis device, wherein the controller is responsive to commands provided from the iontophoresis interface system via the communications port.

39. The iontophoresis system of claim 37 wherein the monitoring subsystem monitors a rate of delivery of the active agent.

40. The iontophoresis system of claim 37 wherein the monitoring subsystem monitors an amount of the active agent delivered by the iontophoresis device.

41. The iontophoresis system of claim 37 wherein the monitoring subsystem monitors a current flow through at least a portion of the iontophoresis device.

42. The iontophoresis system of claim 37 wherein the monitoring subsystem monitors voltage across at least a portion of the iontophoresis device.

43. The iontophoresis system of claim 37 wherein the monitoring subsystem monitors a time during which the iontophoresis device has been at least one of active or inactive.

44. The iontophoresis system of claim 37 wherein the monitoring subsystem monitors a delivery profile of the active agent over a period of time.

45. The iontophoresis system of claim 30 wherein the user interface is operable to produce user generated requests for information indicative of an operational condition of the iontophoresis device and to provide information to the user indicative of the operational condition.

46. The iontophoresis interface system of claim 30 wherein in the user interface is operable to produce user generated requests for information indicative of a rate of transfer of the active agent by the at least one iontophoresis device, and to provide information to the user indicative of the rate of transfer of the active agent by the at least one iontophoresis device.

47. The iontophoresis interface system of claim 30 wherein in the user interface is operable to produce user generated requests for information indicative of an amount of the active agent transferred by the at least one iontophoresis device, and to provide information to the user indicative of the amount of the active agent transferred by the at least one iontophoresis device.

48. The iontophoresis interface system of claim 30 wherein the user interface is operable to produce user generated requests for information indicative of a current flow through at least a portion of the at least one iontophoresis device, and to provide information to the user indicative of the current flow through at least a portion of the at least one iontophoresis device.

49. The iontophoresis interface system of claim 30 wherein the user interface is operable to produce user generated requests for information indicative of a voltage across at least a portion of the at least one iontophoresis device, and to provide information to the user indicative of the voltage across at least a portion of the at least one iontophoresis device.

50. The iontophoresis interface system of claim 30 wherein the user interface is operable to produce user generated requests for information indicative of a time during which the iontophoresis device has been at least one of active or inactive, and to provide information to the user indicative of the time during which the iontophoresis device has been at least one of active or inactive.

51. The iontophoresis interface system of claim 30 wherein in user interface is operable to produce user generated requests for information indicative of a delivery profile of the active agent over a period of time, and to provide information to the user indicative of the delivery profile of the active agent over the period of time.

52. The iontophoresis system of claim 30 wherein the processor is operable to provide commands to the first iontophoresis device from the iontophoresis interface system via the communications port based at least in part on the user generated requests.

53. The iontophoresis system of claim 52 wherein at least some of the commands provided by the iontophoresis interface system to the at least one iontophoresis device are indicative of a delivery rate for delivering active agent to a biological interface from the iontophoresis device.

54. The iontophoresis system of claim 52 wherein at least some of the commands provided by the iontophoresis interface system to the at least one iontophoresis device are indicative of an amount of an active agent to deliver to a biological interface from the iontophoresis device.

55. The iontophoresis system of claim 52 wherein at least some of the commands provided by the iontophoresis interface system to the at least one iontophoresis device are indicative of a current flow through at least a portion of the iontophoresis device.

56. The iontophoresis system of claim 52 wherein at least some of the commands provided by the iontophoresis interface system to the at least one iontophoresis device are indicative of a voltage across at least a portion of the iontophoresis device.

57. The iontophoresis system of claim 52 wherein at least some of the commands provided by the iontophoresis interface system to the at least one iontophoresis device are indicative of a time during which the iontophoresis device is to actively deliver an active agent to a biological interface.

58. The iontophoresis system of claim 52 wherein at least some of the commands provided by the iontophoresis interface system to the at least one iontophoresis device are indicative of a delivery profile for delivering the active agent from the iontophoresis device to the biological interface.

59. The iontophoresis system of claim 30 wherein the communications port comprises at least one antenna element.

60. The iontophoresis system of claim 30 wherein the communications port comprises at least one communications connector.

61. The iontophoresis system of claim 30 wherein the communications path is at least one of a wired network connection or wireless network connection.

62. A method for operating an iontophoresis device, the method comprising:
storing a quantity of an active agent in an active agent reservoir;
delivering at least a portion of the active agent to a biological interface from the iontophoresis device by applying an electrical potential of a first polarity to an active electrode element;
communicating between the iontophoresis device and a remote device via a communications port; and
decrypting commands provided from the remote device via the communications port.

63. The method of claim 62, further comprising:
at least partially controlling the delivery of the active agent to the biological interface from the iontophoresis device upon decrypting the commands provided from the remote device via the communications port.

64. The method of claim 63 wherein communicating between the iontophoresis device and the remote device includes determining whether the commands provided from the remote device via the communications port are authorized, and to at least initially reject any commands that are determined not to be authorized.

65. The method of claim 62 wherein communicating between the iontophoresis device and the remote device comprises receiving user generated operational requests via a user interface operable by a user and providing authorizing requests to the remote device via the communications port based on the user generated operational requests, and upon decrypting the commands provided from the remote device via the communications port in response to the authorizing requests, responding to the commands.

66. The method of claim 65 wherein providing authorizing requests and responding to the commands include at least one of providing wired or wireless authorizing requests.

67. The method of claim 65 wherein providing authorizing requests to the remote device via the communications port includes encrypting the authorizing requests.

68. The method of claim 65 wherein responding to the commands provided from the remote device via the communications port includes determining whether the commands are authorized, and to at least initially reject any commands that are determined not to be authorized.

69. A method operating an interface system to remotely interact with iontophoresis devices useful in delivery of active agents to biological interfaces, the method comprising:
providing a user interface at a location remote from at least some of the iontophoresis devices;
communicating with at least two of the iontophoresis devices; and
providing information about the iontophoresis devices via the user interface.

70. The method of claim 69, further comprising:
producing a number of requests for operational parameter information, wherein communicating with at least two of the iontophoresis devices includes communicating the requests for operational parameter information to the respective iontophoresis device and receiving a response to the request.

71. The method of claim 70 wherein providing information about the iontophoresis devices via the user interface includes providing information based at least in part on the received response to the request.

72. The method of claim 70 wherein producing a number of requests for operational parameter information comprises producing requests for information indicative of a rate of transfer of an active agent by the at least one iontophoresis device and providing information to the user indicative of the rate of transfer of the active agent by the at least one iontophoresis device.

73. The method of claim 70 wherein producing a number of requests for operational parameter information comprises producing requests for information indicative of an amount of an active agent transferred by the at least one iontophoresis device and providing information to the user indicative of the amount of the active agent transferred by the at least one iontophoresis device.

74. The method of claim 70 wherein producing a number of requests for operational parameter information comprises producing requests for information indicative of a current flow through at least a portion of the at least one iontophoresis device and providing information to the user indicative of the current flow through the at least one iontophoresis device.

75. The method of claim 70 wherein producing a number of requests for operational parameter information comprises producing requests for information indicative of a voltage across at least a portion of the at least one iontophoresis device and providing information to the user indicative of the voltage across at least a portion of the at least one iontophoresis device.

76. The method of claim 70 wherein producing a number of requests for operational parameter information comprises producing requests for information indicative of a time during which the iontophoresis device has been at least one of active or inactive and providing information to the user indicative of the time during which the iontophoresis device has been at least one of active or inactive.

77. The method of claim 70 wherein producing a number of requests for operational parameter information comprises producing requests for information indicative of a delivery profile of the active agent over a period of time and providing information to the user indicative of the delivery profile of the active agent over the period of time.

78. The method of claim 69 wherein communicating with at least two of the iontophoresis devices comprises providing at least one of wired or wireless communications.

79. The method of claim 69 wherein communicating with at least two of the iontophoresis devices comprises providing commands to the iontophoresis devices.

80. The method of claim 79 wherein providing commands to the iontophoresis devices comprises providing commands indicative of a delivery rate for delivering an active agent to a biological interface from the iontophoresis device.

81. The method of claim 79 wherein providing commands to the iontophoresis devices comprises providing commands indicative of an amount of an active agent to deliver to a biological interface from the iontophoresis device.

82. The method of claim 79 wherein providing commands to the iontophoresis devices comprises providing commands indicative of a current flow through at least a portion of the iontophoresis device.

83. The method of claim 79 wherein providing commands to the iontophoresis devices comprises providing commands indicative of a voltage across at least a portion of the iontophoresis device.

84. The method of claim 79 wherein providing commands to the iontophoresis devices comprises providing commands indicative of a time during which the iontophoresis device is to actively deliver an active agent to a biological interface.

85. The method of claim 79 wherein providing commands to the iontophoresis devices comprises providing commands indicative of a delivery profile for delivering an active agent from the iontophoresis device to a biological interface.

86. A method for operating an iontophoresis system, the method comprising:
providing communications between a plurality of iontophoresis devices and at least one iontophoresis interface system remotely located from the iontophoresis devices;
operating the iontophoresis devices at least in part on commands received from the iontophoresis interface system; and
at least temporarily storing operational condition information at the iontophoresis interface system, the operational condition information indicative of at least one operating parameter for a number of the iontophoresis devices.

87. The method of claim 86 wherein operating the iontophoresis devices at least in part on commands received from the control system comprises at least partially controlling the delivery of an active agent to a biological interface from the iontophoresis device in response to the commands provided from the iontophoresis interface system via the communications port.

88. The method of claim 87 wherein providing communications between a plurality of iontophoresis devices and at least one iontophoresis interface system remotely located from the iontophoresis devices comprises decrypting the commands provided from the iontophoresis interface system.

89. The method of claim 87 wherein providing communications between a plurality of iontophoresis devices and at least one iontophoresis interface system remotely located from the iontophoresis devices comprises determining whether the commands provided from the iontophoresis interface system are authorized, and to at least initially reject any commands that are determined not to be authorized.

90. The method of claim 87 wherein providing communications between a plurality of iontophoresis devices and at least one iontophoresis interface system remotely located from the iontophoresis devices comprises providing to the iontophoresis interface system requests for authorization to adjust an operational condition of the iontophoresis device.

91. The method of claim 90 wherein requests for authorization to adjust an operational condition of the iontophoresis device are produced in response to user generated operational requests.

92. The method of claim 86, further comprising:
monitoring at least one operational aspect of the iontophoresis device and providing signals indicative of the monitored operational aspect to the iontophoresis interface system.

93. The method of claim 92 wherein monitoring at least one operational aspect of the iontophoresis device includes monitoring a rate of delivery of an active agent from the iontophoresis device.

94. The method of claim 92 wherein monitoring at least one operational aspect of the iontophoresis device includes monitoring an amount of an active agent delivered by the iontophoresis device.

95. The method of claim 92 wherein monitoring at least one operational aspect of the iontophoresis device includes monitoring a current flow through at least a portion of the iontophoresis device.

96. The method of claim 92 wherein monitoring at least one operational aspect of the iontophoresis device includes monitoring voltage across at least a portion of the iontophoresis device.

97. The method of claim 92 wherein monitoring at least one operational aspect of the iontophoresis device includes monitoring a time during which the iontophoresis device has been at least one of active or inactive.

98. The method of claim 92 wherein monitoring at least one operational aspect of the iontophoresis device includes monitoring a delivery profile indicative of a delivery of an active agent by the iontophoresis device over a period of time.

99. The method of claim 86, further comprising:
presenting the operational condition information via a user interface remotely from the iontophoresis devices.

100. The method of claim 86 wherein providing communications between a plurality of iontophoresis devices and at least one iontophoresis interface system remotely located from the iontophoresis devices comprises providing at least one of wired or wireless communications between the iontophoresis devices and the at least one iontophoresis interface system.
